(19) United States
(12) Patent Application Publication
(10) Pub. No.: US 2008/0261875 A1
(43) Pub. Date: Oct. 23, 2008

(54) Method For Treating Cardiovascular Disease

(75) Inventors: Garrett Jay Etgen, Carmel, IN (US); Alexei Kharitonenkov, Zionsville, IN (US)

Correspondence Address: ELI LILLY & COMPANY, PATENT DIVISION, P.O. BOX 6288, INDIANAPOLIS, IN, 46206-6288, US

(73) Assignee: Eli Lilly and Company, Indianapolis, IN (US)

(21) Appl. No.: 11/722,376

(22) PCT Filed: Jan. 9, 2006
(86) PCT No.: PCT/US06/00339

§ 371 Date: Jun. 21, 2007

Related U.S. Application Data
(60) Provisional application No. 60/645,706, filed on Jan. 21, 2005.

(51) Int. Cl. A61K 38/18; A61P 9/10

(52) U.S. Cl. 514/12

(57) ABSTRACT

A method for treating cardiovascular disease comprising administering an effective amount of FGF-21 or an FGF-21 compound to a patient in need thereof.

### BACKGROUND OF THE INVENTION

1. Field of Invention

This invention relates to the use of fibroblast growth factor 21 or compounds thereof for the treatment of cardiovascular disease.

2. Description of the Art

Cardiovascular disease (CVD) is the leading killer in the United States for both men and women among all racial and ethnic groups. CVD includes a number of conditions affecting the structures or function of the heart. These conditions can include arteriosclerosis and atherosclerosis, stroke, abnormal heart rhythms or arrythmias, heart muscle disease, aorta disease, heart failure, and vascular disease.

Well established risk factors for CVD are elevated low-density lipoprotein (LDL) cholesterol, elevated triglyceride levels and low levels of high-density lipoprotein (HDL) cholesterol (W. B. Kannel, et al., American Heart Journal, 148(1): 16-26, (2004); C. M. Ballantyne et al., American Heart Journal, 146(2): 227-233 (2003)). In addition, apolipoprotein CIII (apoCIII) is emerging as an important risk factor for CVD. High apoCIII levels prolong the duration of VLDL and LDL as well as block the breakdown of triglycerides, all CVD promoting effects. In fact, one study has shown nearly all the adverse effects of high triglycerides to be due to elevated apoCIII (Sung-Joon Lee et al., Arteriosclerosis, Thrombosis, and Vascular Biology 2003; 23:853). Also, in individuals on lipid-lowering medications, a high apoCIII level remains as an independent CVD risk despite improved overall lipid profiles.

Adiponectin, a polypeptide predominantly secreted by adipocytes, has been shown to be inversely associated with the cardiovascular risk factors mentioned above, and is positively related to HDL cholesterol levels (Scherer P E, et al., J Biol. Chem. 270:26746-26749, (1995); Hotta K, et al., Arterioscler Thromb Vasc Biol.; 20:1595-1599, (2000)). Adiponectin levels are significantly reduced in obese subjects (Y. Arita et al., Biochem Biophys Res Commun 257:79-83, 1999), as well as, in patients with some of the disease states associated with obesity, such as type 2 diabetes and coronary artery disease (N. Ouchi et al, Circulation 100:2473-2476, 1999). Thus, low levels of adiponectin may be considered another risk factor associated with CVD.

Fibroblast growth factors are polypeptides widely expressed in developing and adult tissues (Baird et al., Cancer Cells, 3:239-243, 1991) and play crucial roles in multiple physiological functions including angiogenesis, mitogenesis, pattern formation, cellular differentiation, metabolic regulation and repair of tissue injury (McKeehan et al., Prog. Nucleic Acid Res. Mol. Biol. 59:135-176, 1998). According to the published literature, the FGF family now consists of at least twenty-three members, FGF-1 to FGF-23 (Reuss et al., Cell Tissue Res. 313:139-157 (2003).

Studies have shown that fibroblast growth factors FGF-1, FGF-2, FGF-4 and FGF-5, induce therapeutic angiogenesis, which represents a complex attempt to relieve inadequate blood flow by the directed growth and proliferation of blood vessels (Rissanen et al, FASEB J, 17(1):100-2, (2003)). In CVD, patients with refractory angina and lower extremity intermittent claudication seem most amenable to early tests of therapeutic angiogenesis (R J Aviles, et al., British Journal of Pharmacology 140:637-646, (2003)). The results of these studies have offered promise for new treatment strategies for various ischemic diseases and the use of various FGF polypeptides has prompted investigators and clinicians alike to reconsider the complexity of therapeutic angiogenesis (Ng Y S, et al. Curr Control Trials Cardiovasc Med. 2(6):278-285, 2001).

Fibroblast growth factor-21 (FGF-21)(Nishimura et al., Biochimica et Biophysica Acta, 1492:203-206, 2000; WO01/36640; and WO01/18172) has been described as a treatment for ischemic vascular disease, wound healing, and diseases associated with loss of pulmonary, bronchia or alveolar cell function and numerous other disorders. Additionally, FGF-21 has been shown to stimulate glucose-uptake in mouse 3T3-L1 adipocytes, and to decrease fed and fasting blood glucose in ob/ob and db/db mice in a dose-dependant manner, providing the basis for the use of FGF-21 as a therapy for treating type 2 diabetes and obesity (WO03/011213).

In contrast to the typical angiogenic effect of FGF-like polypeptides described above, FGF-21 was shown to significantly improve the lipid profile and several cardiovascular risk factors in diabetic rhesus monkeys, therefore establishing a novel treatment of CVD with FGF-21. Although many pharmaceutical therapies are currently available for the treatment of various aspects of CVD, there is a need for more effective therapies to reduce the morbidity and mortality associated with this disease. Thus, FGF-21 provides this need in treating CVD by lowering LDL, ApoCIII, or triglyceride levels and raising HDL or adiponectin levels in patients in need of such treatment, consequently, reducing the morbidity and mortality associated with CVD.

### SUMMARY OF THE INVENTION

The present invention provides a method for treating a patient with cardiovascular disease comprising: administering to said patient a therapeutically effective amount of FGF-21 or an FGF-21 compound sufficient to achieve in said patient at least one of the following modifications: a reduction of LDL, a reduction of ApoCIII, an increase in HDL, or in increase in adiponectin.

### DETAILED DESCRIPTION OF THE INVENTION

For purposes of the present invention, as disclosed and claimed herein, the following terms are as defined below.

Human FGF-21 is a 208 amino acid polypeptide containing a 27 amino acid leader sequence. Human FGF-21 has ˜79% amino acid identity to mouse FGF-21 and ˜80% amino acid identity to rat FGF-21. Human FGF-21 and analogs, muteins, or derivatives of human FGF-21 or an alternative mammalian FGF-21 polypeptide sequence could be readily used for the uses described herein.

The mature human 181 amino acid FGF-21 polypeptide is shown below (SEQ ID NO:1):

```
1 10
His Pro Ile Pro Asp Ser Ser Pro Leu Leu Gln Phe
                                                        20
Gly Gly Gln Val Arg Gln Arg Tyr Leu Tyr Thr Asp
                                        30
Asp Ala Gln Gln Thr Glu Ala His Leu Glu Ile Arg
                        40
Glu Asp Gly Thr Val Gly Gly Ala Ala Asp Gln Ser
        50 60
Pro Glu Ser Leu Leu Gln Leu Lys Ala Leu Lys Pro
                                                                        70
Gly Val Ile Gln Ile Leu Gly Val Lys Thr Ser Arg
                                                        80
Phe Leu Cys Gln Arg Pro Asp Gly Ala Leu Tyr Gly
                                        90
Ser Leu His Phe Asp Pro Glu Ala Cys Ser Phe Arg
                        100
Glu Leu Leu Leu Glu Asp Gly Tyr Asn Val Tyr Gln
        110 120
Ser Glu Ala His Gly Leu Pro Leu His Leu Pro Gly
                                                                        130
Asn Lys Ser Pro His Arg Asp Pro Ala Pro Arg Gly
                                                        140
Pro Ala Arg Phe Leu Pro Leu Pro Gly Leu Pro Pro
                                        150
Ala Leu Pro Glu Pro Pro Gly Ile Leu Ala Pro Gln
                        160
Pro Pro Asp Val Gly Ser Ser Asp Pro Leu Ser Met
        170 180
Val Gly Pro Ser Gln Gly Arg Ser Pro Ser Tyr Ala
Ser
```

The corresponding DNA sequence coding for the mature human 181 amino acid FGF-21 polypeptide is (SEQ ID NO:2):

CACCCCATCCCTGACTCCAGTCCTCTCCTGCAATTCGGGGGCCAAGTCCG
GCAGCGGTACCTCTACACAGATGATGCCCAGCAGACAGAAGCCCACCTGG
AGATCAGGGAGGATGGGACGGTGGGGGGCGCTGCTGACCAGAGCCCCGAA
AGTCTCCTGCAGCTGAAAGCCTTGAAGCCGGGAGTTATTCAAATCTTGGG
AGTCAAGACATCCAGGTTCCTGTGCCAGCGGCCAGATGGGGCCCTGTATG
GATCGCTCCACTTTGACCCTGAGGCCTGCAGCTTCCGGGAGCTGCTTCTT
GAGGACGGATACAATGTTTACCAGTCCGAAGCCCACGGCCTCCCGCTGCA
CCTGCCAGGGAACAAGTCCCCACACCGGGACCCTGCACCCCGAGGACCAG
CTCGCTTCCTGCCACTACCAGGCCTGCCCCCCGCACTCCCGGAGCCACCC
GGAATCCTGGCCCCCCAGCCCCCCGATGTGGGCTCCTCGGACCCTCTGAG
CATGGTGGGACCTTCCCAGGGCCGAAGCCCCAGCTACGCTTCC

The FGF-21 useful in the methods of the present invention is preferably human FGF-21. Additionally, the methods of the present invention include the use of FGF-21 analogs, FGF-21 muteins, and FGF-21 derivatives hereinafter collectively known as FGF-21 compounds. FGF-21 compounds have sufficient homology to FGF-21 such that the compound has the ability to bind to the FGF-21 receptor and initiate a signal transduction pathway resulting in glucose uptake stimulation and lowering LDL, triglycerides, or ApoCIII levels and increasing HDL or adiponectin levels. For example, FGF-21 compounds can be tested for glucose uptake activity using a cell-based assay such as that described in Example 1 and tested for effects on lipid profiles in the ob/ob mouse assay as described in Example 2.

A human FGF-21 mutein is defined as comprising human FGF-21 in which at least one amino acid of the wild-type mature protein has been substituted by another amino acid. Examples of FGF-21 muteins are described in U.S. patent applications 60/528,582, 60/606,805, and 60/606,830, herein incorporated by reference. Generally speaking, a mutein possesses some modified property, structural or functional, of the wild-type protein. For example, the mutein may have enhanced or improved physical stability in concentrated solutions (e.g., less hydrophobic mediated aggregation), while maintaining a favorable bioactivity profile. The mutein may possess increased compatibility with pharmaceutical preservatives (e.g., m-cresol, phenol, benzyl alcohol), thus enabling the preparation of a preserved pharmaceutical formulation that maintains the physiochemical properties and biological activity of the protein during storage. The mutein may have reduced O-glycosylation when expressed in yeast. The mutein may have less deamindation when compared to wild type FGF-21. As used herein, these terms are not limiting, it being entirely possible that a given mutein has one or more modified properties of the wild-type protein.

An FGF-21 compound also includes a “FGF-21 derivative” which is defined as a molecule having the amino acid sequence of FGF-21 or an FGF-21 analog or mutein, but additionally having a chemical modification of one or more of its amino acid side groups, α-carbon atoms, terminal amino group, or terminal carboxylic acid group. A chemical modification includes, but is not limited to, adding chemical moieties, creating new bonds, and removing chemical moieties. Examples of FGF-21 derivatives are described in U.S. patent applications 60/553,765 and 60/570,908, herein incorporated by reference.

Modifications at amino acid side groups include, without limitation, acylation of lysine ε-amino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Furthermore, one or more side groups, or terminal groups, may be protected by protective groups known to the ordinarily-skilled protein chemist. The α-carbon of an amino acid may be mono- or dimethylated.

“Adiponectin”, also known as Acrp30 or AdipoQ, is a protein hormone produced and secreted exclusively by adipocytes (fat cells) that regulates the metabolism of lipids and glucose. Adiponectin influences the body's response to insulin. Adiponectin also has anti-inflammatory effects on the cells lining the walls of blood vessels. High blood levels of adiponectin are associated with a reduced risk of heart attack.

“Apolipoprotein C-III” or apoCIII, is a marker of triglyceride-rich lipoproteins. ApoCIII is synthesised in the liver. It inhibits lipoprotein lipase and modulates the uptake of triglyceride-rich particles by LDL receptor-related protein. ApoCIII concentrations are higher in patients with CVD compared with that in control patients.

“LDL” stands for “low density lipoprotein”. Most of the cholesterol in the blood comes from LDL. Elevated LDL cholesterol levels is a major risk factor for CVD.

“VLDL” stands for “very low density lipoprotein” and is composed mostly of cholesterol, with little protein. VLDL is often called “bad cholesterol” because it deposits cholesterol on the walls of arteries. Increased levels of VLDL are associated with CVD.

“HDL” stands for “high density lipoprotein” Increased HDL cholesterol levels are associated with a lower risk of CVD.

“Triglycerides” are the chemical form in which most fat exists in food as well as in the body. They're also present in blood plasma and, in association with cholesterol, form the plasma lipids. Excess triglycerides in plasma is called hypertriglyceridemia. It is linked to the occurrence of CVD.

Fibroblast growth factors have been reported in the scientific literature as treatments for ischemic vascular disease, wound healing, and diseases associated with loss of pulmonary, bronchia or alveolar cell function and similar disorders. The treatment of ischemic vascular disease with fibroblast growth factors has focused on angiogenesis, which occurs by a concerted series of events initiated by the release of the growth factors in ischemic tissue. In other words, angiogenesis induced by fibroblast growth factors creates vessels for ischemic coronary artery disease and peripheral vascular disease.

In contrast, the present invention documents the effect of FGF-21 or FGF-21 compounds on various risk factors associated with CVD, not on angiogenesis associated with ischemic disease. Unexpectedly, FGF-21 or FGF-21 compounds do not induce angiogenesis but rather lower LDL, triglyceride, and/or apoCIII levels and elevate HDL and/or adiponectin levels, all biomarkers associated with CVD. Thus the present invention establishes a novel use of FGF-21 or FGF-21 compounds for the treatment of CVD in patients in need of such treatment.

The FGF-21 administered according to this invention may be generated and/or isolated by any means known in the art such as described in Sambrook et al., Molecular Cloning: A Laboratory Manual, Cold Spring Harbor Laboratory Press, NY (1989).

Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend, for example, on the nature of the production process used for FGF-21.

FGF-21 or FGF-21 compounds may be formulated according to known methods to prepare pharmaceutically useful compositions. A desired formulation would be one that is a stable lyophilized product that is reconstituted with an appropriate diluent or an aqueous solution of high purity with optional pharmaceutically acceptable carriers, preservatives, excipients or stabilizers [Remington's Pharmaceutical Sciences 16th edition (1980)]. The FGF-21 of the present invention may be combined with a pharmaceutically acceptable buffer, and the pH adjusted to provide acceptable stability, and a pH acceptable for administration.

For parenteral administration FGF-21 or FGF-21 compounds are formulated generally, in a unit dosage injectable form (solution, suspension, or emulsion), with a pharmaceutically acceptable carrier. Preferably, one or more pharmaceutically acceptable anti-microbial agents may be added. Phenol, m-cresol, and benzyl alcohol are preferred pharmaceutically acceptable anti-microbial agents.

Optionally, one or more pharmaceutically acceptable salts may be added to adjust the ionic strength or tonicity. One or more excipients may be added to further adjust the isotonicity of the formulation. Glycerin, sodium chloride, and mannitol are examples of an isotonicity adjusting excipient.

“Pharmaceutically acceptable” means suitable for administration to a human. A pharmaceutically acceptable formulation does not contain toxic elements, undesirable contaminants or the like, and does not interfere with the activity of the active compounds therein.

If subcutaneous or an alternative type of administration is used, the FGF-21 compounds may be derivatized or formulated such that they have a protracted profile of action.

A “therapeutically effective amount” of FGF-21 or an FGF-21 compound is the quantity that results in a desired effect without causing unacceptable side-effects when administered to a subject. A desired effect can include an amelioration of symptoms associated with the disease or condition, a delay in the onset of symptoms associated with the disease or condition, and increased longevity compared with the absence of treatment. In particular, the desired effect is a reduction of LDL, apoCIII and/or triglyceride levels and an increase in HDL and/or adiponectin levels associated with CVD.

The pharmaceutical compositions of the FGF-21 or FGF-21 compounds in the present invention may be administered by any means that achieve the generally intended purpose: to treat CVD. For example, administration may be by oral or parenteral administration. The term “parenteral” as used herein refers to modes of administration that include intravenous, intramuscular, intraperitoneal, intrasternal, subcutaneous, and intraarticular injection and infusion. The dosage administered will be dependent upon the age, health, and weight of the recipient, kind of concurrent treatment, if any, frequency of treatment, and the nature of the effect desired. Compositions within the scope of the invention include all compositions wherein FGF-21 is present in an amount that is effective to achieve the desired medical effect for treatment of CVD. While individual needs may vary from one patient to another, the determination of the optimal ranges of effective amounts of all of the components is within the ability of the clinician of ordinary skill.

Those skilled in the art can readily optimize pharmaceutically effective dosages and administration regimens for therapeutic compositions comprising FGF-21 or FGF-21 compounds, as determined by good medical practice and the clinical condition of the individual patient. A typical dose range for FGF-21 or FGF-21 compounds will range from about 0.01 mg per day to about 1000 mg per day for an adult. Preferably, the dosage ranges from about 0.1 mg per day to about 100 mg per day, more preferably from about 1.0 mg/day to about 10 mg/day. Most preferably, the dosage is about 1-5 mg/day.

The appropriate dose of FGF-21 or FGF-21 compounds administered will result in a reduction of LDL, apoCIII and/or triglyceride levels and an increase in HDL and/or adiponectin levels associated with CVD.

Alternatively, FGF-21 or FGF-21 compounds may be administered twice weekly at a dose range from about 0.01 mg per dose to about 1000 mg per dose for an adult. Preferably, the dosage ranges from about 0.1 mg per dose to about 100 mg per dose, more preferably from about 1.0 mg per dose to about 10 mg per day. Most preferably, the dosage is about 1-5 mg per dose.

In another alternative, FGF-21 or FGF-21 compounds may be administered once weekly at a dose range from about 0.01 mg per dose to about 1000 mg per dose for an adult. Preferably, the dosage ranges from about 0.1 mg per dose to about 100 mg per dose, more preferably from about 1.0 mg per dose to about 10 mg per dose. Most preferably, the dosage is about 1-5 mg per dose.

In another aspect of the present invention, FGF-21 or FGF-21 compounds for use as a medicament for the treatment of CVD is contemplated.

Having now described the present invention in detail, the same will be more clearly understood by reference to the following examples, which are included herewith for purposes of illustration only and are not intended to be limiting of the invention.

All patents and publications referred to herein are expressly incorporated by reference.

### Preparation 1

### Expression and Purification of FGF-21 in Yeast

An expression system for production of FGF-21 or FGF-21 compounds is yeast, such as Pichia pastoris, Pichia methanolica or Saccharomyces cerevisiae. For production in Pichia pastoris, a commercially available system (Invitrogen, Carlsbad, Calif.) uses vectors with the powerful AOX1 (alcohol oxidase) promoters to drive high-level expression of recombinant proteins. Alternatively, vectors that use the promoter from the GAP gene (glyceraldehyde-3-phosphate dehydrogenase) are available for high level constitutive expression. The multi-copy Pichia expression vectors allow one to obtain strains with multiple copies of the gene of interest integrated into the genome. Increasing the number of copies of the gene of interest in a recombinant Pichia strain can increase protein expression levels.

### EXAMPLE 1

### Glucose Uptake in Mouse 3T3-L1 Adipocytes

3T3-L1 cells are obtained from the American Type Culture Collection (ATCC, Rockville, Md.). Cells are cultured in growth medium (GM) containing 10% calf serum in Dulbecco's modified Eagle's medium. For standard adipocyte differentiation, two days after cells reach confluency (referred as day 0), the cells are exposed to differentiation medium (DM) containing 10% fetal bovine serum, 5 μg/ml of insulin, 1 μM dexamethasone, and 0.5 μM isobutylmethylxanthine, for 48 h and then are exposed to medium containing 10% fetal bovine serum, 5 μg/ml insulin for an additional 48 h. Cells are then maintained in post differentiation medium containing 10% fetal bovine serum.

Glucose Transport Assay—FGF-21 or FGF-21 compounds are added to the differentiated 3T3-L1 cells in 96 well plates at 0, 0.016, 0.08, 0.4, 2, 10, or 50.0 nM. The plates are incubated at 37° C. for 72 hours.

Hexose uptake, as assayed by the accumulation of 2-deoxy-D-[¹⁴C]glucose, is measured as follows: 24 hours prior to the assay, the wells are rinsed twice with PBS and DMEM (high glucose, 1% antibiotic/antimycotic solution, 2 mM glutamine), 0.1% BSA plus FGF-21 is added. The plates are incubated at 37° C. for 72 hours. The cells are then washed twice with KRP buffer (136 mM NaCl, 4.7 mM KCl, 10 mM NaPO₄, 0.9 mM CaCl₂, 0.9 mM MgSO₄, 0.1% BSA, pH 7.4), and then KRP buffer containing 1% BSA, 2 deoxy-D-glucose, 100 μM, 0.1 μCi/well 2-deoxy-D-[¹⁴C]glucose is added and the plates are incubated at 37° C. for one hour. Cytochalasin B is added to stop further glucose uptake. Uptake is measured on a Microbeta plate reader.

In vitro potency is normalized to the in vitro activity of wild-type FGF-21, which is given a designation of 1.0 and used as a positive control. The in vitro potency of various FGF-21 compounds compared to wild-type FGF-21 is shown in Table 1.

TABLE 1

| | Expression | In vitro |
|---|---|---|
| FGF-21 Mutein | System | Potency* |
| Wild-type | E. coli | 1.0 |
| des-HPIP Truncated Wild- | Yeast | 0.9 |
| type** | | |
| des-HPIP L118C, A134C | Yeast | 0.2 |
| des-HPIP L118C, A134C, | Yeast | 0.2 |
| S167A | | |
| des-HPIP L118C-A134C- | Yeast | 0.25 |
| N121S | | |
| Fc-L-FGF-21*** | Yeast | 0.15 |

*potency is a relative value based on the activity of E. coli produced wild-type FGF-21
**truncated by 4 amino acids at the N-terminus
***N-terminus of FGF-21 fused to the C-terminus of the fusion protein via a linker peptide, (Gly-Gly-Gly-Gly-Ser)₃.

### EXAMPLE 2

### Ob/ob Mouse Model

The Ob/ob mouse model is an animal model for hyperglycemia, insulin resistance and obesity. Male ob/ob mice are used to monitor plasma glucose levels and triglyceride levels after treatment with FGF-21 or FGF-21 compounds. Male ob/ob mice (7 weeks old) are treated with FGF-21 or FGF-21 compounds at 5 μg/day or 3 μg/day. FGF-21 or FGF-21 compounds are administered s.c. in 0.1 ml and compared to the s.c. vehicle control (0.9% NaCl, 0.1 ml/mouse).

The animals are dosed daily for 14 days. Blood glucose levels are measured daily, 1 hour post dosing, using a standard protocol, Table 2. Triglyceride levels are measured on day 14. As shown in Table 3, FGF-21 and various FGF-21 compounds significantly lower triglyceride levels in ob/ob mice.

TABLE 2

| | Plasma Glucose levels |
|---|---|
| FGF-21 Mutein | as % of Control |
| Wild-type | 62% |
| L118C-A134C | 70% |
| L118C-A134C-S167A | 62% |
| R77E | 63% |

TABLE 3

| | Triglyceride |
|---|---|
| FGF-21 Mutein | Levels (mg/dL) |
| Vehicle Control | 210 |
| Wild-type | 116*** |
| L118C-A134C | 137** |
| L118C-A134C-S167A | 153* |
| R77E | 125** |

P value vs. vehicle control:
*p ≦ 0.05;
**p ≦ 0.02;
***p ≦ 0.001

### EXAMPLE 3

### Dose Escalation Study in Diabetic Rhesus Monkeys

A dose escalation study in diabetic rhesus monkeys is done to monitor the following parameters after treatment with FGF-21: plasma glucose levels, triglyceride levels, LDL levels, and HDL levels. The dosing protocol is as follows: Vehicle dosing of all monkeys begins on Day 1 and continues for 14 days. On day 14 through day 27, FGF-21 is administered, s.c., at 30 μg/kg. At day 29 through day 42, FGF-21 is administered, s.c., at 100 μg/kg. At day 43 through day 56, FGF-21 is administered, s.c., at 300 μg/kg. From day 57 to day 84 the animals are not dosed (washout period). On days 14, 28, 42, 56, and 84, an IVGTT (Intravenous glucose tolerance test) assay is performed. On days 1, 7, 14, 21, 28, 35, 42, 49, 56, 70, and 84, blood was drawn and assayed for the above listed parameters. The parameter values on Day 1 are used as the baseline calculation. The parameter values determined on days 7 and 14 are averaged as the vehicle control; days 21 and 28 are averaged for the 30 μg/kg dose level; days 35 and 42 are averaged for the 100 μg/kg dose level; days 48 and 56 are averaged for the 300 μg/kg dose level; and, day 84 is used for the washout value. Assays utilized to determine the various parameters measured are well known in the art.

Plasma glucose levels determined as described above are shown in Table 4. Plasma glucose levels are lowered by FGF-21 treatment in a dose dependent manner. In addition, an effect is still apparent after the 21 day washout period.

TABLE 4

| | | | % Reduction |
|---|---|---|---|
| FGF-21 Dose Level | Mean | StdErr | from Baseline |
| Baseline | 119.2 | 8.2 | 0 |
| 30 μg/kg | 104 | 10.2 | 10 |
| 100 μg/kg | 82.8 | 10 | 28 |
| 300 μg/kg | 70.5 | 4.8 | 39 |
| Washout | 94 | 11.3 | 18 |

Plasma triglyceride levels determined as described above are shown in Table 5. Plasma triglyceride levels are lowered by FGF-21 treatment in a dose dependent manner. In addition, an effect is still apparent after the 21 day washout period.

TABLE 5

| | | | % Reduction |
|---|---|---|---|
| FGF-21 Dose Level | Mean | StdErr | from Baseline |
| Baseline | 626 | 215.2 | 0 |
| 30 μg/kg | 413.4 | 145.6 | 33 |
| 100 μg/kg | 298.5 | 107.5 | 52 |
| 300 μg/kg | 193.7 | 82.9 | 69 |
| Washout | 390 | 146 | 38 |

Plasma HDL levels determined as described above are shown in Table 6. Plasma samples are taken on the days indicated, assayed, and mean values are calculated. Plasma HDL levels are raised by FGF-21 treatment in a dose dependent manner. In addition, an effect is still apparent after the 21 day washout period.

TABLE 6

| FGF-21 Dose Level | | % Increase |
|---|---|---|
| (day sample taken) | Mean (mg/dl) | from Baseline |
| Baseline (1, 7, 14) | 28.8 | 0 |
| 30 μg/kg (21, 28) | 37.2 | 29 |
| 100 μg/kg (35, 42) | 46.0 | 60 |
| 300 μg/kg (49, 56) | 51.6 | 79 |
| Washout (70, 84) | 35.0 | 22 |

Plasma LDL levels determined as described above are shown in Table 7. Plasma samples are taken on the days indicated, assayed, and mean values are calculated. Plasma LDL levels are lowered by FGF-21 treatment in a dose dependent manner. In addition, an effect is still apparent after the 21 day washout period.

TABLE 7

| FGF-21 Dose Level | | % Decrease |
|---|---|---|
| (day sample taken) | Mean (mg/dl) | from Baseline |
| Baseline (1, 7, 14) | 83.0 | 0 |
| 30 μg/kg (21, 28) | 75.1 | 10 |
| 100 μg/kg (35, 42) | 66.4 | 20 |
| 300 μg/kg (49, 56) | 59.9 | 28 |
| Washout (70, 84) | 68.3 | 18 |

### EXAMPLE 4

### Rules-Based Medicine

Rules-Based Medicine (RBD) [Austin, Tex.] is a service laboratory which provides Multi-Analyte Profile (MAP) testing. MAPs are high-density, quantitative immunoassay panels for mice, rats, monkeys and humans that allow the alteration in biomarker patterns to be identified. MAPs provide a comprehensive evaluation of the protein expression patterns indicative of response to disease, drugs, and the environment. By comparing samples of experimental subjects with controls, relevant patterns emerge.

CVD biomarkers are assayed in plasma samples from the diabetic rhesus monkeys of Example 3 utilizing the RBD technology. Essentially, microspheres impregnated with fluorescent dyes are coated with reagents that bind with target substances in the blood. A system of lasers and computers recognizes when a reaction takes place, indicating the presence and concentration of a particular protein. The RBD analysis for CVD biomarkers in the diabetic rhesus monkey plasma samples from the FGF-21 treated animals shows an approximate 50% reduction of apoCIII (39.8 baseline to 20.1 final) and an approximate two fold increase in adiponectin (2.7 baseline to 4.6 final), thereby demonstrating a positive impact of FGF-21 on biomarkers associated with CVD.

We claim:

1. A method for treating cardiovascular disease in a patient in need thereof comprising administering to said patient a therapeutically effective amount of FGF-21 or an FGF-21 compound sufficient to achieve in said patient at least one of the following modifications: a reduction of LDL, a reduction of apoCIII, an increase in HDL, or in increase in adiponectin.

2. The method of claim 1 wherein said modification is a reduction in LDL.

3. The method of claim 1 wherein said modification is a reduction in apoCIII.

4. The method of claim 1 wherein said modification is an increase in HDL.

5. The method of claim 1 wherein said modification is an increase in adiponectin.

6. (canceled)

7. (canceled)

8. (canceled)

9. (canceled)

10. (canceled)


SEQUENCE LISTING (2 sequences)

SEQ ID NO: 1 - protein, 181 aa, homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - DNA, 543 bp, homo sapiens
caccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcc